(19) United States
(12) Patent Application Publication
(10) Pub. No.: US 2013/0189769 A1
(43) Pub. Date: Jul. 25, 2013

(54) BIOCHEMICAL SENSOR

(71) Applicants: YUAN-CHEN LEE, TAIPEI CITY (TW); CHIN-HUI SU, NEW TAIPEI CITY (TW)

(72) Inventors: YUAN-CHEN LEE, TAIPEI CITY (TW); CHIN-HUI SU, NEW TAIPEI CITY (TW)

(73) Assignee: RAPHA BIO LTD., NEW TAIPEI CITY (TW)

(21) Appl. No.: 13/355,521

(22) Filed: Jan. 21, 2012

(51) Int. Cl. G01N 27/00; C12M 1/40

(52) U.S. Cl. 435/287.9; 422/82.01

(57) ABSTRACT

A biochemical sensor includes a substrate with a printing surface, an intermediate layer, and a cover. The printing surface includes a reactive film, two electrode films, a conductive electrode film, and a referential electrode film. The conductive electrode film is arranged between the two electrode films. Each ends of the two electrode films contacts with the reactive film. One end of the conductive electrode film, proximate to the reactive film, contacts with one of the two electrode films. The referential electrode film and one of the two electrode films are arranged on the relative two sides of the printing surface. One end of the referential electrode film extends to contact with the reactive film. The intermediate layer has a notch corresponding to the reactive film, and the cover has an test slot corresponding to the notch. The intermediate layer and the cover are sequentially covered on the substrate.

### BACKGROUND OF THE INVENTION

1. Field of the Invention

The instant disclosure relates to a sensor; particularly to a biochemical sensor capable of performing a higher accuracy through averaging a value from a plurality of readings measured after a reaction occurred between a test sample and a reactive film.

2. Description of Related Art

Typically, patients have to take their blood tests in hospitals for regular physical checkups. However, such periodical trips from home to hospital for health checkups are both time and energy consuming, resulting in physical and mental exhaustions for patients.

Presently, there are already portable or domestic-grade blood test devices developed to provide convenience for patients to keep track of their physical conditions, and the method for operating the devices are simple. By instilling the test sample on the test strip for reaction with the reactive film, a plurality of resistance values can be obtained. Then the test strip is inserted into a blood detection device for display of the readings which concern the health condition of the user. Therefore, patients can monitor their physical conditions at all times by utilizing these blood test devices at their preferred locations without the need of going to hospitals or organizations for blood tests. Thereby, providing convenience for the users.

Shown in FIGS. 1 and 2, which show a test strip 1a of the prior art. The test strip 1a includes a cover 11a, an intermediate layer 12a, and a substrate 13a. The intermediate layer 12a and the cover 11a are attached sequentially to the substrate 13a. There are two electrode films 131a and one conductive electrode film 132a printed on the substrate 13a. Each of the two electrode films 131a has a reactive film 134a arranged on the respective ends thereof One end of the conductive electrode film 132a is in connection to one of the two electrode films 131a. For such structural designs, the distance between the two respective ends of each of the two electrode films 131a, where a reactive film 134a is arranged on one end thereof, will be too long. Therefore, in cases when blood instilled into the test strip 1a is insufficient, the two reactive films 134a arranged on the two respective ends of the electrode films 131a will receive insufficient amount of blood for reaction. In other words, a value may have been calculated by the blood detection device even during the travel of the blood within the two electrode films 131a. Thereby, resulting in an inaccurate reading as the insufficient of blood has lead to determination errors. In addition, carbon films are often utilized for reactive films 134a. In general, if carbon films are solely utilized as reactive mediums, an unstable current value may be obtained under operation. For example, when the concentrations of the user's blood differ, a current value with a relatively higher variation coefficient may be obtained. In other words, a larger current value will be obtained if the blood concentration is higher. Conversely, a smaller current value will be obtained if the blood concentration is lower. Thus, the instability of the current may also lead to determination errors of the readings.

To address the above issues, the inventor strives via industrial experience and academic research to present the instant disclosure, which can effectively improve the limitations described above.

### SUMMARY OF THE INVENTION

The object of the instant disclosure is to provide a biochemical sensor capable of analyzing the reactivity and blood amount of the test sample instilled on the reactive film through a referential electrode film, and make appropriate corrections when required. Thereby, increasing the accuracy of determining the pathological analysis of the test sample.

In order to achieve the aforementioned objects, according to an embodiment of the instant disclosure, a biochemical sensor is provided, which includes a substrate with a printing surface formed thereon. The printing surface includes a reactive film, two electrode films, a conductive electrode film, and a referential electrode film. The conductive electrode film is arranged between the two electrode films, and one end of the conductive electrode film, proximate to the reactive film, is in connection to one of the two electrode films. The referential electrode film and one of the two electrode films are arranged on the relative two sides of the printing surface. The reactive film has at least one first metallic reactive film, one non-metallic reactive film, and one second metallic reactive film. One end of the referential electrode film extends to establish contact with the first metallic reactive film. Each of the ends of the two electrode films are in contact respectively with the second metallic reactive film and the non-metallic reactive film. An intermediate layer is provided which has at least one notch corresponding to the reactive film. And a cover is provided which has at least one test slot corresponding to the notch. The intermediate layer and the cover are sequentially covered on the substrate. The intermediate layer is arranged and inter-attached between the cover and the substrate.

Based on the above, the instant disclosure relates to a biochemical sensor, where the referential electrode film is printed on the substrate. Through the analysis by the referential electrode film on reactivity and blood amount of the test sample instilled on the reactive film, and make appropriate corrections when required, the accuracy of determining the pathological analysis of the test sample can be increased. In addition, the distance between the two ends of the two electrode films and the referential electrode film, where one end of the aforementioned films have a reactive film arranged thereon respectively, are shortened. Thereby, the distance travelled by the test samples are concurrently shortened. Thus, increasing the accuracy of the readings. Furthermore, the constitution of the metallic and non-metallic reactive films provides a more stable current output value to effectively reduce the time spend on detecting.

In order to understand the characteristics of the creative and technical content, please refer to the following detailed description about the creation and charts, but it provides a reference type and description only and is not used to restrict the creation.

### BRIEF DESCRIPTION OF THE DRAWINGS

FIG. 1 shows a three-dimensional exploded view of a biological sensor in accordance to a prior art;

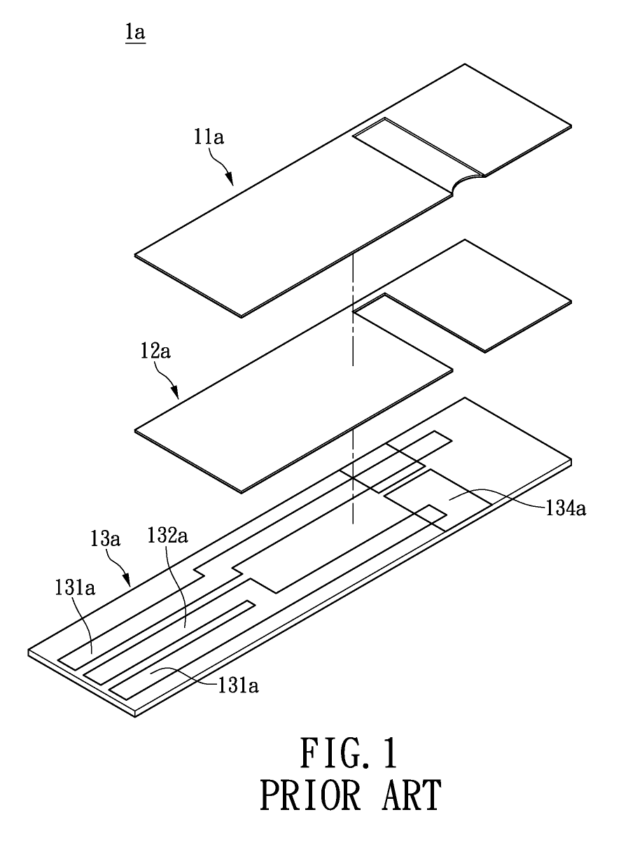

FIG. 2 shows a three-dimensional assembled view of a biological sensor in accordance to a prior art;

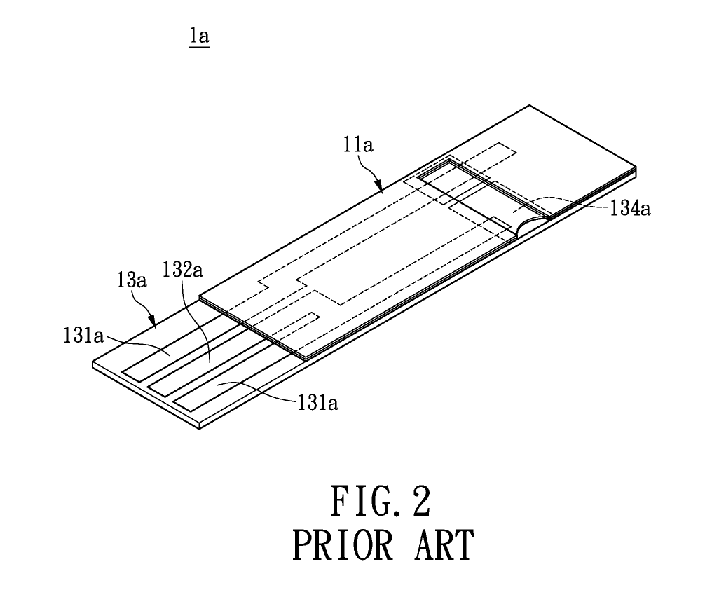

FIG. 3 shows a three-dimensional exploded view of a biological sensor in accordance to the first embodiment of the instant disclosure;

FIG. 4 shows a three-dimensional assembled view of a biological sensor in accordance to the first embodiment of the instant disclosure;

FIG. 5 shows a three-dimensional view of a biological sensor under operation in accordance to the first embodiment of the instant disclosure;

FIG. 6 shows a three-dimensional exploded view of a biological sensor in accordance to the second embodiment of the instant disclosure.

### DETAILED DESCRIPTION OF THE PREFERRED EMBODIMENTS

Unless stated otherwise, all mentioned quantities are not intended to be limiting.

### The First Embodiment

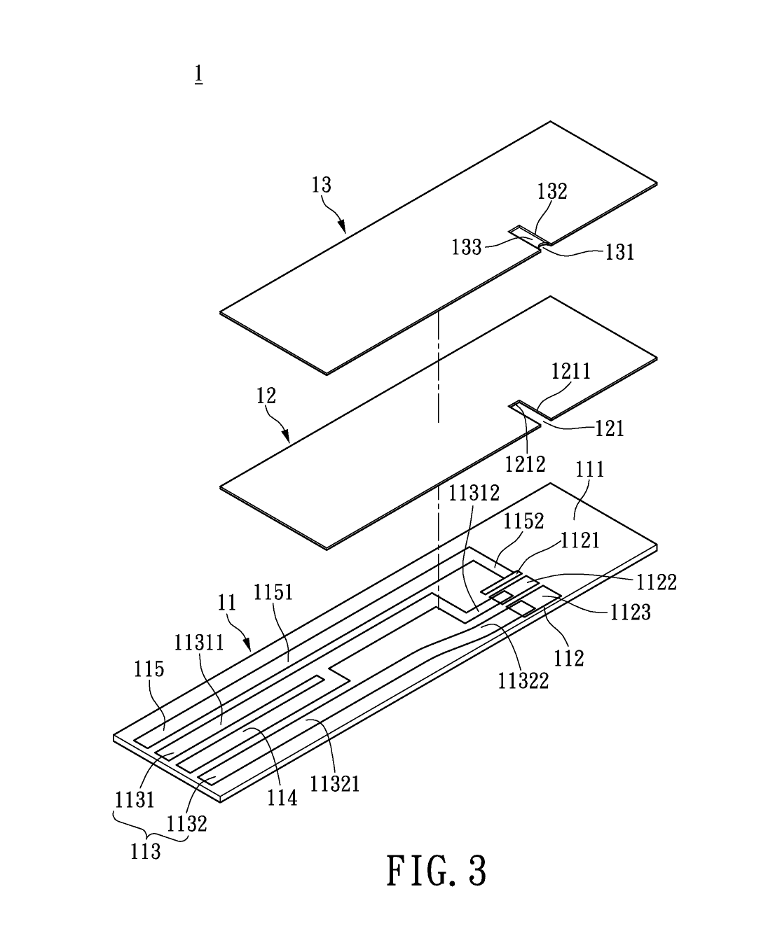

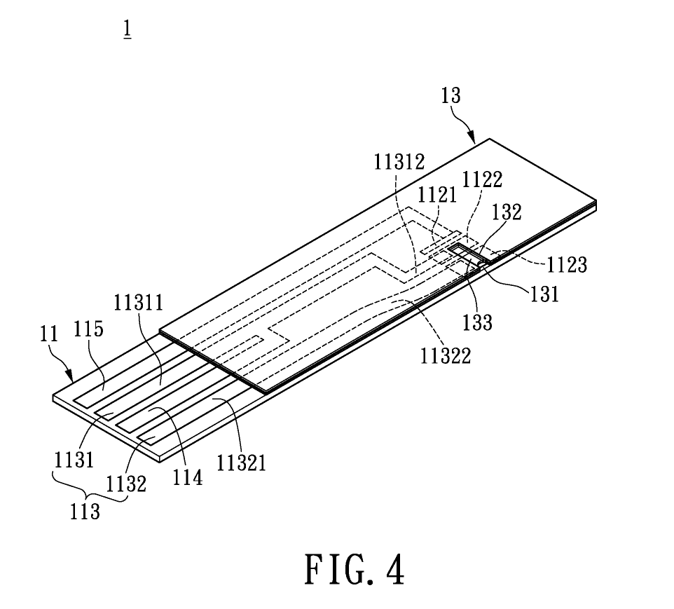

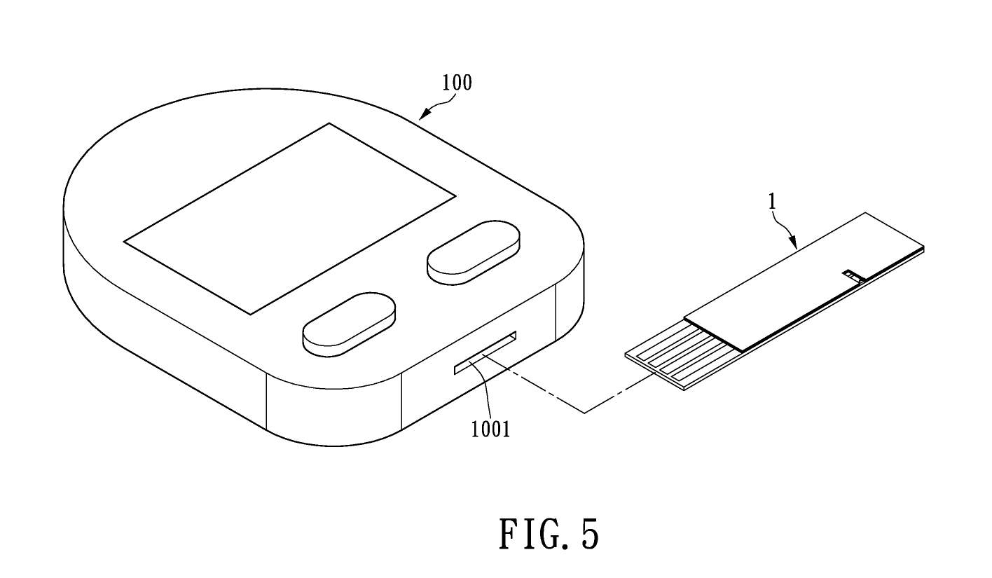

Please refer to FIGS. 3-5. FIGS. 3-4 show, respectively, the three-dimensional exploded and assembled views of a biological sensor in accordance to the first embodiment of the instant disclosure; where FIG. 5 shows a three-dimensional view of a biological sensor under operation in accordance to the first embodiment of the instant disclosure. An embodiment of the instant disclosure provides a biochemical sensor 1, which can be inserted into an all-in-one type of test device 100, and the test device 100 has an insertion slot 1001. The sheet-like biochemical sensor 1 can be inserted into the insertion slot 1001 for analysis of the transmitted signals within the biochemical sensor 1. Therefore, measurements (such as glucose, triglycerides, etc.) from the test samples (e.g. blood) can be obtained to determine the health condition of the user. The biochemical sensor 1 includes a substrate 11, an intermediate layer 12, and a cover 13. With the orientation of the figures provided, the biochemical sensor 1 is formed of the assembly of the cover 13, which is arranged on the top layer, the substrate 11, which is arranged on the bottom layer, and the intermediate layer 12 arranged therebetween. Generally, the test samples of the users are distributed to the reacting area of the substrate 11 through the siphon principle.

The substrate 11 has a printing surface 111 thereon, which has a reactive film 112, two electrode films 113, a conductive electrode film 114, and a referential electrode film 115. The substrate 11 of the instant disclosure utilizes the method of printing to form the reactive film 112, the two electrode films 113, the conductive electrode film 114, and the referential electrode film 115 on the printing surface 111 of the substrate 11. Furthermore, dry reactive enzymes are disposed on the reactive film 112. The electrode films 113, the conductive electrode film 114, and the referential electrode film 115 can be made of, but not limited to, carbon (C), platinum (Pt), or silver (Ag) electrodes. When the biochemical sensor 1 is inserted into the test device 100, the referential electrode film 115, and the two electrode films 113 will test to determine the non-ideal testing data before generating a plurality of measurements. Then, an average value will be calculated from the measurements to increase accuracy.

Please refer to FIGS. 3 and 5. The conductive electrode film 114 is arranged between the two electrode films 113. The conductive electrode film 114 and the test device 100 are electrically connected to provide starting function for the test device 100. One end of the conductive electrode film 114, proximate to the reactive film 112, is connected to one of the two electrode films 113. The referential electrode film 115 and one of the two electrode films 113 are arranged on the relative two sides of the printing surface 111. The reactive film 112 has a first metallic reactive film 1121, a non-metallic reactive film 1122, and a second metallic reactive film 1123, where the three reactive films 1121, 1122, and 1123 are equally spaced apart from each other to keep a predetermined distance therebetween. One end of the referential electrode film 115 extends to establish contact with the first metallic reactive film 1121. Each end of the two electrode films 113 are respectively in contact with the non-metallic reactive film 1122, and the second metallic reactive film 1123. The referential electrode film 115 is capable of analyzing reactivity and blood amount for the test sample on the reactive film 112. Furthermore, the referential electrode film 115 can make appropriate corrections for the measurements generated by the test device 100.

To provide further explanations, the first and the second metallic reactive films 1121, 1123 of the instant disclosure is preferably made of silver, where the non-metallic reactive film 1122 is preferably made of carbon. Additionally, through experimental reports show that the constitution of utilizing silver for the first and the second metallic films 1121,1123, and carbon for the non-metallic reactive film 1122 will provide a higher stability for the output current. In other words, by adopting blood samples of identical concentration to compare with conventional biochemical sensors, the arrangement and materials utilized in the first, second metallic reactive films 1121, 1123, and the non-metallic reactive film 1122 of the instant disclosure are capable of obtaining a higher current output value. Thereby, determination errors due to different blood concentrations which may further lead to higher variation coefficient of the current values can be avoided. However, the materials utilized in the first, second metallic reactive films 1121, 1123, and the non-metallic reactive film 1122 are only preferences for the instant embodiment. Practically, other constitutions utilizing different metallic and non-metallic reactive films can also achieve the same effect of producing a stable current value. Therefore, the materials utilized in the first, second metallic reactive films 1121, 1123, and the non-metallic reactive film 1122 are not restricted thereto. Thus, the materials and the combination thereof utilized by any ordinary skilled person in the relevant art based on existing prior art should fall within the claim coverage of the instant disclosure.

Generally, the reactive film 112 is includes the first metallic reactive film 1121, the non-metallic reactive film 1122, and the second metallic reactive film 1123. The reactive films 1121, 1122, 1123 are spaced apart from each other to provide a more stable current output value, and a reduced detecting time. Thus, the detecting time can be ideally shortened to less than 30 seconds for obtaining the measurements.

The intermediate layer 12 is a transparent insulation layer, disposed on the printing surface 111 of the substrate 11. The intermediate layer 12 defines at least one notch 121 on an elevated portion thereof, which is in correspondence to the reactive film 112. Therefore, the elevated portion restricts the reactive enzymes within the notch 121 to prevent escaping therefrom. Please note that the intermediate layer 12 of the instant disclosure can be made of polyethylene (PE), polyvinyl chloride (PVC) or other polymer materials, where the materials are not restricted thereto.

Please refer to FIG. 3. The cover 13 of the instant disclosure is hydrophilic, which has at least one test slot 131 corresponding to the notch 121. Thereby, the test sample can be instilled onto the reactive film 112 via the test slot 131. The intermediate layer 12 and the cover 13 are sequentially covered on the substrate 11. To provide further explanations, the intermediate layer 12 is arranged and inter-attached between the substrate 11 and the cover 13 to cooperatively form a stacked structure. In other words, after the substrate 11 has gone through the printing process, the intermediate layer 12 will be attached to the substrate 11, prior to the attachment of the cover 13 to the intermediate layer 12 in a mating shape. The means of attaching the stacked structure includes, but not limited to, using a single-sided tape, or a liquid adhesive, etc.

Please refer to FIGS. 3 and 5. The two electrode films 113, the conductive electrode film 114, and the referential electrode film 115 are not covered completely by the intermediate layer 12 and the cover 13. Therefore, the films 113, 114, 115 are exposed partially to the surrounding for insertion into the test device 100.

The referential electrode film 115 has a first referential electrode extended portion 1151 and a second referential electrode extended portion 1152, where a bent structure is formed therebetween. Furthermore, one end of the second referential electrode extended portion 1152 establishes contact with the reactive film 112.

The two electrode films 113 are designed to include a common electrode film 1131, and a measuring electrode film 1132. The common electrode film 1131 is arranged between the referential electrode film 115 and the conductive electrode film 114. One end of the common electrode film 1131 is in contact with the non-metallic reactive film 1122, while one end of the measuring electrode film 1132 is in contact with the second metallic reactive film 1123. The common electrode film 1131 has a first common electrode extended portion 11311, and a second common electrode extended portion 11312, where a bent structure is formed therebetween.

The measuring electrode film 1132 has a first measuring electrode extended portion 11321, and a second measuring electrode extended portion 11322, where a ramp structure is formed therebetween. Furthermore, the first referential electrode extended portion 1151, the first common electrode extended portion 11311, the conductive electrode film 114, and the first measuring electrode extended portion 11321 are arranged in parallel.

One end of the second common electrode extended portion 11312, away from the first common electrode extended portion 11311, is proximate to the second measuring electrode extended portion 11322. Therefore, the area required for arranging the reactive film 112 can be minimized. In comparison to conventional reactive films where a larger area is required for the arrangement thereof, the distance for the test sample to travel in the instant disclosure is shorter. Therefore, readings may be generated by conventional detection device even before the test samples are distributed entirely within the reactive film. Thus, the readings may be inaccurate which are unworthy to refer. Furthermore, the distance between the second common electrode extended portion 11312 and the second measuring electrode extended portion 11322 is shortened in the instant disclosure. Thereby, upon instilling the test samples onto the reactive film 112 through the test slot 131, the test samples can be distributed entirely to the reactive film 112 through the siphon principle. Thus, a reading of a smaller deviation can be achieved to increase accuracy.

The first referential electrode extended portion 1151, the first common electrode extended portion 11311, the conductive electrode film 114, and the first measuring electrode extended portion 11321 are equally spaced apart from each other.

The cover 13 has at least one slot 132 formed peripherally, and a transparent layer 133 formed on the slot 13. The test slot 131 is formed on the mouth of the transparent layer 133. The transparent layer 133 is provided to observe the distribution of the test sample on the reactive film 112. The transparent layer 133 is spaced apart from the printing surface 111 of the substrate 11. The transparent layer 133, the notch 121 and the printing surface 111 of the substrate 11 cooperatively enclose to define a sample channel (not labeled), where the test slot 131 is arranged thereon. Therefore, the sample channel flows in a vertical direction with respect to the substrate 11.

The notch 121 is enclosed by a first width 1211 and a second width 1212, where the width of the first width 1211 is greater than that of the second width 1212. Furthermore, the sample channel is defined in a rectangular shape.

### The Second Embodiment

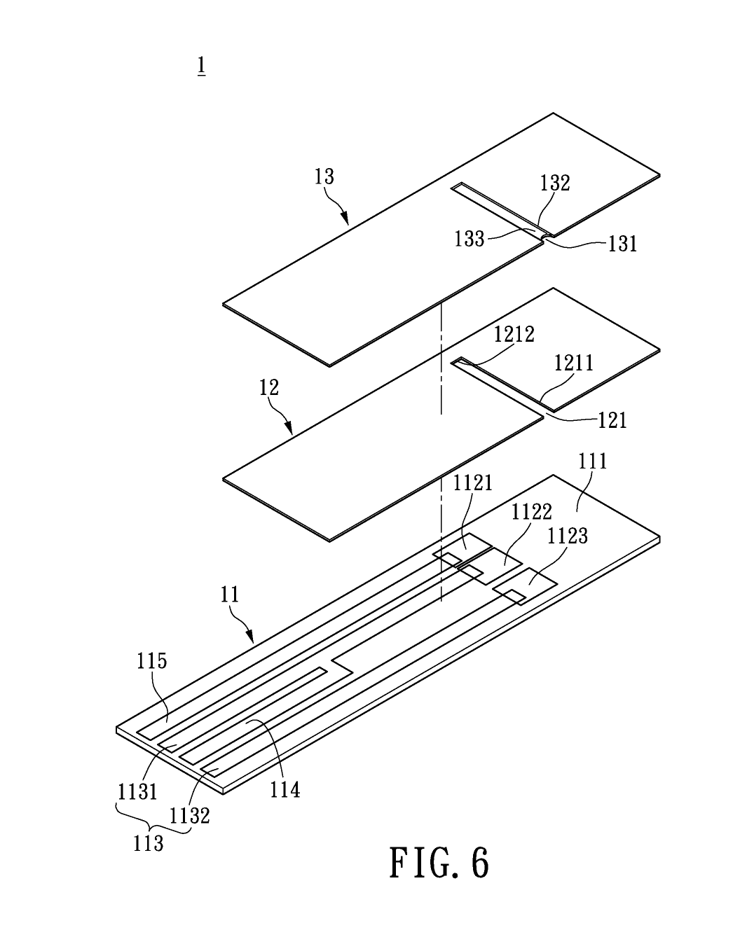

Please refer to FIG. 6 which shows a three-dimensional exploded view of a biological sensor in accordance to the second embodiment of the instant disclosure. The instant embodiment has improved from the preceding embodiment through a simplified layout of the two electrode films 113, and the referential electrode film 115 on the substrate 11. For instance, the two electrode films 113 and the referential electrode film 115 are arranged in parallel, where they extend straightly for the formation thereof One end of the common electrode 1131 is in contact to the non-metallic reactive film 1122, while one end of the measuring electrode film 1132 is in contact with the second metallic reactive film 1123. Thus, the simplified layout of the two electrode films 113 and the referential electrode film 115 on the substrate 11 can reduce the production cost of printing the aforementioned films. However, in comparison to the preceding embodiment, the distances between the two ends of the two electrode film 113, and the two ends of the referential electrode film 115, where the reactive films 112 are arranged on one of the ends, are relatively longer. In other words, the distance required to be travelled by the test sample is longer. Therefore, more test samples are needed for distribution onto the entire reactive film 112. Furthermore, the materials included in the reactive films 112, and the layouts (referring to the layout of the non-metallic reactive film 1122 between the first and the second metallic reactive films 1121, 1123) of the printing surface 111 enable a stable output of the current value. Thereby, providing the same effect of increasing accuracy of the readings.

Please refer to FIGS. 3 and 5. In order to provide a better understanding of the instant disclosure, the manufacturing process of the biological sensor 1 in accordance to the instant disclosure is illustrated in the following descriptions.

Fastening the substrate 11 on a fastening fixture (not shown);

Printing the reactive film 112, the two electrode films 113, the conductive electrode film 114, and the referential electrode film 115 concurrently on the substrate 11;

Attaching the intermediate layer 12, located above the reactive film 112, to the substrate 11 by mean of gluing;

Spraying or squeezing the liquid reactive enzyme onto the reactive film 112;

Drying the reactive enzyme in a low temperature for at least 25 minutes till the reactive enzyme transforms into the solid state;

Attaching the cover 13 to the intermediate layer 12 in a mating shape by utilizing means of gluing;

Trimming the substrate 11 to a desired strip-like structure.

For in use, the test sample is instilled onto the reactive film 112. When electrochemical reaction occurs between the reactive enzyme on the reactive film 112 and the test sample, a plurality of transmitting signals will be detected by the two electrode films 113, and the referential electrode film 115. The transmitting signals will then be transmitted to the test device 100 for calculation of a plurality of measurements (e.g. glucose) within the test object. If a small difference is detected by the plurality of measurements, an average value will be obtained as the blood glucose value of the test object. Conversely, if a large difference is detected between the plurality of measurements, it is deemed that the amount of blood under testing may be inadequate.

Please note, the directional terms mentioned in the instant disclosure such as: top, bottom, left, right, front, and back are only references for the attached figures. Therefore, the directional terms are merely for explanation purposes and are not intended to restrict the instant disclosure.

Based on the above, the instant disclosure relates to a biochemical sensor, which has a referential electrode film printed on the substrate. The biochemical sensor is capable of analyzing the reactivity and blood amount of the test sample instilled on the reactive film through the referential electrode film, and make appropriate corrections when required. Thereby, increasing the accuracy of determining the pathological analysis of the test sample. In addition, the distances between the two ends of the two electrode films, and the two ends of the referential electrode film, where the reactive films are arranged on one of the ends, are shortened to reduce the distance travelled by the test sample. Thereby, increasing the accuracy of the readings. Furthermore, the constitution of the metallic and non-metallic reactive films also provides a more stable current output value to effectively reduce the time spend on detecting.

The descriptions illustrated supra set forth simply the preferred embodiments of the instant disclosure; however, the characteristics of the instant disclosure are by no means restricted thereto. All changes, alternations, or modifications conveniently considered by those skilled in the art are deemed to be encompassed within the scope of the instant disclosure delineated by the following claims.

What is claimed is:

1. A biochemical sensor, comprising:
a substrate, which has a printing surface, wherein the printing surface includes a reactive film, two electrode films, a conductive electrode film, and a referential electrode film, the conductive electrode film is arranged between the two electrode films, one end of the conductive electrode film, proximate to the reactive film, is connected to one end of the two electrode films, the referential electrode film and one of the two electrode films are arranged on the relative two sides of the printing surface, the reactive film has at least one first metallic reactive film, one non-metallic reactive film, and one second metallic reactive film, one end of the referential electrode film extends to establish contact with the first metallic reactive film, while each end of the two electrode films are in contact respectively with the non-metallic reactive film and the second metallic reactive film;
an intermediate layer which has at least one notch in correspondence to the reactive film; and
a cover which has at least one test slot in correspondence to the notch, the intermediate layer and the cover are attached sequentially to the substrate, the intermediate layer is arranged and inter-attached between the cover and the substrate.

2. The biochemical sensor according to claim 1, wherein the two electrode films, the conductive electrode film, and the referential electrode film are partially exposed to the surrounding through the intermediate layer and the cover.

3. The biochemical sensor according to claim 1, wherein the first metallic reactive film, the non-metallic reactive film, and the second metallic reactive film are equally spaced apart from each other.

4. The biochemical sensor according to claim 1, wherein the cover has at least one slot formed peripherally, and a transparent layer formed on the slot, and wherein the test slot is formed on the mouth of the transparent layer.

5. The biochemical sensor according to claim 4, wherein the transparent layer is spaced apart from the printing surface of the substrate, and wherein the transparent layer, the notch and the printing surface of the substrate cooperatively enclose to define a sample channel, wherein the sample channel flows in a vertical direction with respect to the substrate.

6. The biochemical sensor according to claim 5, wherein the notch is enclosed by a first width and a second width, wherein the width of the first width is greater than that of the second width, and the sample channel is defined in a rectangular shape.

7. The biochemical sensor according to claim 6, wherein the test slot is defined above the sample channel.

8. The biochemical sensor according to claim 1, wherein the intermediate layer is an insulation layer.

9. The biochemical sensor according to claim 1, wherein the referential electrode film has a first referential electrode extended portion and a second referential electrode extended portion, the two electrode films are designed to include a common electrode film, and a measuring electrode film, wherein the common electrode film is arranged between the referential electrode film and the conductive electrode film, and wherein the common electrode film has a first common electrode extended portion, and a second common electrode extended portion, the measuring electrode film has a first measuring electrode extended portion, and a second measuring electrode extended portion, wherein the first referential electrode extended portion, the first common electrode extended portion, the conductive electrode film, and the first measuring electrode extended portion are arranged in parallel.

10. The biochemical sensor according to claim 9, wherein a bent structure is formed between the second referential electrode extended portion and the first referential electrode extended portion, wherein a bent structure is formed between the second common electrode extended portion and the first common electrode extended portion, and wherein a bent structure is formed between the second measuring electrode extended portion and the first measuring electrode extended portion.

11. The biochemical sensor according to claim 9, wherein one end of the second common electrode extended portion, away from the first common electrode extended portion, is proximate to the second measuring electrode extended portion.

12. The biochemical sensor according to claim 9, wherein the first referential electrode extended portion, the first common electrode extended portion, the conductive electrode film, and the first measuring electrode extended portion are equally spaced apart from each other.
